(19) United States
(12) Patent Application Publication
(10) Pub. No.: US 2026/0027034 A1
(43) Pub. Date: Jan. 29, 2026

(54) COSMETIC COMPOSITION CONTAINING MICRONEEDLES OF POLYMER

(71) Applicant: JNTL CONSUMER HEALTH KABUSHIKI KAISHA, Tokyo (JP)

(72) Inventor: Mao WAKABAYASHI, Tokyo (JP)

(21) Appl. No.: 19/120,555

(22) PCT Filed: Oct. 13, 2023
(86) PCT No.: PCT/JP2023/037257

§ 371 Date: Apr. 11, 2025

(30) Foreign Application Priority Data
Oct. 27, 2022 (JP) 2022-172382

(51) Int. Cl. A61K 8/73; A61Q 19/02; A61Q 19/08

(52) U.S. Cl. CPC A61K 8/735; A61Q 19/02; A61Q 19/08; A61K 2800/52

(57) ABSTRACT

Provided is a cosmetic composition in which microneedles including a water soluble polymer are uniformly and stably dispersed and combined in a formulation including a cosmetic component.

The cosmetic composition includes an oil gel 2 including a cosmetic component, an oil gelling agent, and an oil component, and microneedles 3 including a water soluble polymer, the microneedles 3 being dispersed in the oil gel 2. The oil component includes hydrocarbon, ester oil, and silicone oil in an amount of 0 to 10.0 weight %, based on the total cosmetic composition, and the oil gelling agent is present in an amount of 0.5 to 20.0 weight %, based on the total cosmetic composition.

### TECHNICAL FIELD

The present invention relates to a cosmetic composition including a cosmetic component and microneedles.

### BACKGROUND ART

Cosmetic compositions containing cosmetic components and hyaluronic acid are known (Patent Documents 1, 2). Formulation example 2 of Patent Document 1 describes an oil-in-water emulsified cosmetic containing retinol and sodium hyaluronate as cosmetic components. Formulation Example 4 of Patent Document 2 describes a skin cosmetic (emulsion) containing retinol and acetylated sodium hyaluronate as cosmetic components. In a cosmetic composition containing cosmetic components and hyaluronic acid, the molecular weight of hyaluronic acid dissolved in the cosmetic composition is large, and hyaluronic acid cannot penetrate the skin. Therefore, hyaluronic acid in the cosmetic composition is expected to have only a moisturizing effect on the skin surface and is not expected to promote the effects of the cosmetic components.

A needle patch in which microneedles containing hyaluronic acid are arranged on the surface of a substrate is known (Patent Documents 3, 4). The area where microneedles are arranged on the needle patch is small. Therefore, a sufficient amount of cosmetic components to achieve the desired effect cannot be incorporated into the needle patch.

Further, a non-aqueous composition is known, in which microneedles formed from a mixed solution containing hyaluronic acid and a bioactive ingredient are dispersed in an oil phase component (Patent Document 5). In the non-aqueous composition of Patent Document 5, the bioactive ingredient is contained in the microneedles. Since the microneedles are microscopic, a sufficient amount of bioactive ingredient to obtain the desired effect cannot be incorporated into the microneedles.

### CITATION LIST

### Patent Literature

PTL 1: International Publication WO 2020/137785

PTL 2: Japanese Patent Application Publication 2013-189397

PTL 3: Japanese Patent Application Publication 2020-164432

PTL 4: Japanese Patent Application Publication 2009-201956

PTL 5: PCT Application Japanese Translation Publication 2018-535236

### SUMMARY OF INVENTION

### Technical Problem

In order to obtain a cosmetic composition that contains a sufficient amount of cosmetic components to achieve a desired effect and also to promote the effect of the cosmetic components, it is desirable to combine microneedles into a formulation that contains cosmetic components.

However, when microneedles containing water soluble polymers such as hyaluronic acid are added to formulations in the form of emulsified creams, emulsions, or water-soluble gels containing cosmetic components such as retinol, the microneedles dissolve. Therefore, microneedles are unstable in formulations in the form of emulsified creams, emulsions, and water-soluble gels.

When microneedles containing water soluble polymers such as hyaluronic acid are added to formulations in the form of oil containing cosmetic components such as retinol, the microneedles may precipitate. Therefore, the microneedles may not be uniformly dispersed in the formulation in the form of oil.

The present invention was made in view of the aforementioned problem, and the objective of the invention is to provide a cosmetic composition in which microneedles, including a water soluble polymer are uniformly and stably dispersed and combined in a formulation including a cosmetic component.

In a preferred embodiment, it is an objective to provide a cosmetic composition that stably contains a cosmetic component.

Further, in a preferred embodiment, it is an objective to provide a smooth cosmetic composition without stickiness.

### Solution to Problem

The inventors of the present invention diligently studied to achieve the above-mentioned objectives and came to the knowledge that by adjusting respective content amounts of oil gelling agent and oil component that constitute an oil gel, microneedles including a water soluble polymer such as hyaluronic acid can be uniformly and stably dispersed and combined in an oil gel including a cosmetic component such as retinol.

The present invention was made based on the above knowledge, and includes the following configurations.

### Configuration 1

A cosmetic composition including:
an oil gel including a cosmetic component, an oil gelling agent, and an oil component, and
microneedles including a water soluble polymer, the microneedles being dispersed in the oil gel,
in which the oil component includes hydrocarbon, ester oil, and silicone oil in an amount of 0 to 10.0 weight %, based on the total cosmetic composition, and
   in which the oil gelling agent is present in an amount of 0.5 to 20.0 weight %, based on the total cosmetic composition.

### Configuration 2

The cosmetic composition of configuration 1, in which the microneedles are present in an amount of 0.1 to 10.0 weight %, based on the total cosmetic composition.

### Configuration 3

The cosmetic composition of configuration 1 or 2, in which the cosmetic component is present in an amount of 0.01 to 5.0 weight %, based on the total cosmetic composition.

### Configuration 4

The cosmetic composition of any of configurations 1 to 3, in which the oil gelling agent is present in an amount of 5 to 15.0 weight %, based on the total cosmetic composition.

### Configuration 5

The cosmetic composition of any of configurations 1 to 4, in which the cosmetic component includes at least one selected from a group consisting of an anti-wrinkle component, a whitening component, and a germicidal component.

### Configuration 6

The cosmetic composition of configuration 5, in which the anti-wrinkle component includes retinol.

### Configuration 7

The cosmetic composition of configuration 6, in which the oil gel further includes a stabilizing agent including a phenolic antioxidant (preferably, at least one of butylated hydroxyanisole (BHA) and dibutylhydroxytoluene (BHT)).

### Configuration 8

The cosmetic composition of any of configurations 1 to 7, in which the oil gelling agent includes at least one of a polymeric oil gelling agent (particularly, hydrogenated (styrene/isoprene) copolymer) and silica.

### Configuration 9

The cosmetic composition of any of configurations 1 to 8, in which the silicone oil includes dimethicone.

### Configuration 10

The cosmetic composition of any of configurations 1 to 9, in which the hydrocarbon includes at least one selected from a group consisting of olefin hydrocarbon and paraffin hydrocarbon.

### Configuration 11

The cosmetic composition of any of configurations 1 to 10, in which the ester oil includes at least one selected from an ester of a fatty acid and a polyalcohol (particularly, at least one of pentaerythrityl tetraethylhexanoate and tri(caprylic/capric) glyceryl) and carbonic diester (particularly, dicaprylyl carbonate).

### Configuration 12

The cosmetic composition of any of configurations 1 to 11, in which the hydrocarbon includes at least one of hydrogenated polydecene and isohexadecane.

### Configuration 13

The cosmetic composition of any of configurations 1 to 12, in which the ester oil includes at least one selected from dicaprylyl carbonate, pentaerythrityl tetraethyl-hexanoate, and tri(caprylic/capric) glyceryl.

### Configuration 14

The cosmetic composition of any of configurations 1 to 13, in which the oil gelling agent includes at least one of hydrogenated (styrene/isoprene) copolymer and silica.

### Configuration 15

The cosmetic composition of any of configurations 1 to 14, in which the water soluble polymer includes hyaluronic acid or a salt thereof.

### Configuration 16-1

A cosmetic composition including, based on the total cosmetic composition:
an oil gel including:
(a) 0.01 to 5.0 weight % of retinol,
(b) 0.5 to 15.0 weight % of an oil gelling agent including at least one of a polymeric oil gelling agent (particularly, hydrogenated (styrene/isoprene) copolymer) and silica, and
(c) an oil component including 0 to 10.0 weight % of silicone oil (particularly, dimethicone), 0 to 95 weight % of hydrocarbon (particularly, at least one of hydrogenated polydecene and isohexadecane), and 0 to 95 weight % of ester oil (particularly, at least one selected from dicaprylyl carbonate, pentaerythrityl tetraethyl-hexanoate, and tri(caprylic/capric) glyceryl); and
0.1 to 10.0 weight % of microneedles including hyaluronic acid or a salt thereof, the microneedles being dispersed in the oil gel.

### Configuration 16-2

A cosmetic composition including, based on the total cosmetic composition:
an oil gel including:
(a) 0.01 to 5.0 weight % of retinol,
(b) 5 to 15.0 weight % of an oil gelling agent including at least one of a polymeric oil gelling agent (particularly, hydrogenated (styrene/isoprene) copolymer) and silica, and
(c) an oil component including 0 to 10.0 weight % of silicone oil (particularly, dimethicone), 40 to 90 weight % of hydrocarbon (particularly, at least one of hydrogenated polydecene and isohexadecane), and 0 to 45 weight % of ester oil (particularly, at least one selected from dicaprylyl carbonate, pentaerythrityl tetraethyl-hexanoate, and tri(caprylic/capric) glyceryl); and
0.1 to 10.0 weight % of microneedles formed of hyaluronic acid or a salt thereof, the microneedles being dispersed in the oil gel,
in which the cosmetic composition is anhydrous.

### Configuration 16-3

The cosmetic composition of configuration 16-1 or 16-2, in which the oil gel further includes a stabilizing agent including a phenolic antioxidant (preferably, at least one of butylated hydroxyanisole (BHA) and dibutylhydroxytoluene (BHT)).

### Configuration 16-4

The cosmetic composition of configuration 16-3, in which the stabilizing agent is present in an amount of 0.01 to 5 weight % (more preferably 0.01 to 1 weight %, further preferably 0.05 to 1 weight %), based on the total cosmetic composition.

### Advantageous Effects of Invention

As mentioned above, the cosmetic composition of the present invention includes an oil gel including a cosmetic component, an oil gelling agent, and an oil component, and microneedles including a water soluble polymer, the microneedles being dispersed in the oil gel, the oil component including hydrocarbon, ester oil, and silicone oil in an amount of 0 to 10.0 weight %, based on the total cosmetic composition, and the oil gelling agent is present in an amount of 0.5 to 20.0 weight %, based on the total cosmetic composition. Therefore, a cosmetic composition can be obtained in which microneedles including a water soluble polymer are uniformly and stably dispersed and combined in an oil gel including a cosmetic component.

Further, another cosmetic composition according to the present invention includes, based on the total cosmetic composition: an oil gel including (a) 0.01 to 5.0 weight % of retinol, (b) 5 to 15.0 weight % of an oil gelling agent including at least one of a polymeric oil gelling agent and silica, and (c) an oil component including 0 to 10.0 weight % of silicone oil, 40 to 90 weight % of hydrocarbon, and 0 to 45 weight % of ester oil; and 0.1 to 10.0 weight % of microneedles formed of hyaluronic acid or a salt thereof, the microneedles being dispersed in the oil gel, where the cosmetic composition is anhydrous. Therefore, a cosmetic composition can be obtained in which microneedles formed of hyaluronic acid or a salt thereof are uniformly and stably dispersed and combined in an oil gel including retinol.

### BRIEF DESCRIPTION OF DRAWINGS

FIG. 1 is a schematic diagram showing the cosmetic composition of the embodiment.

FIG. 2 is a schematic diagram of the microneedles used in the examples and comparative examples.

### DESCRIPTION OF EMBODIMENTS

The embodiment of the present invention is specifically explained below. The embodiment given below is one of the embodiments upon embodying the present invention and is not intended to limit the present invention within such a scope.

### A. Cosmetic Composition

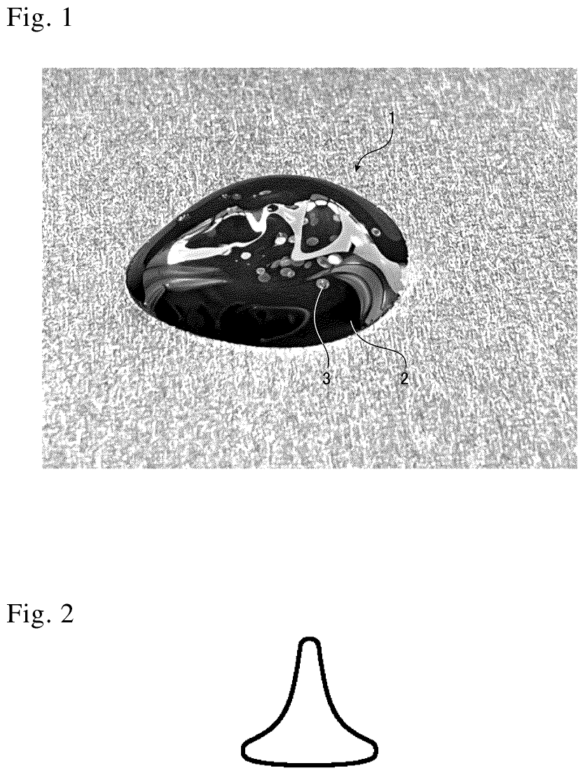

FIG. 1 is a schematic diagram showing the cosmetic composition of this embodiment. The cosmetic composition 1 of this embodiment includes an oil gel 2 and microneedles 3 dispersed in the oil gel 2. Preferably, the cosmetic composition 1 of this embodiment is anhydrous.

### 1. Oil Gel

The oil gel includes a cosmetic component, an oil gelling agent, and an oil component. Preferably, the oil gel further includes a stabilizing agent.
(1) Cosmetic component

The cosmetic component is preferably present in an amount of 0.01 to 5.0 weight %, based on the total cosmetic composition. When the amount is less than the above range, the effect cannot be obtained; and the amount exceeding the above range may cause irritation, etc.

Examples of cosmetic components included in the oil gel include whitening components, anti-wrinkle components, and germicidal components.

Examples of the whitening components include placenta extract, magnesium L-ascorbyl phosphate, sodium L-ascorbyl phosphate, kojic acid, arbutin, L-ascorbic acid 2-glucoside, ellagic acid, 4-n-butylresorcinol, chamomilla ET, S linoleic acid, tranexamic acid, 4-methoxysalicylic acid potassium salt, 3-O-ethyl ascorbate, disodium adenosine monophosphate, 5,5′-dipropyl-biphenyl 2,2′-diol, ascorbyl tetra-2-hexyldecanoate EX, nicotinic acid amide, cetyl tranexamic acid hydrochloride, dex-panthenol W, phenylethyl resorcinol, and sclareolide.

Examples of the anti-wrinkle components include acetylglucosamine, astaxanthin, turmeric extract, retinol or retinol derivatives, tretinoin, retinol acetate, vitamin A palmitate, and beech extract.

Examples of germicidal components include sodium dl-α-tocopheryl phosphate, o-cymen-5-ol, phellodendron bark extract, and salicylic acid.

In some embodiments, the cosmetic components include retinol or retinol derivatives. Examples of retinol derivatives include, without limitation, other retinoids including retinal, retinoic acid, retinyl acetate, retinyl palmitate, and amine derivatives.

In certain embodiments, the cosmetic components include retinol. In one embodiment, retinol is present in an amount of preferably 0.01 to 2 weight %, more preferably 0.01 to 1.0 weight %, and further preferably 0.01 to 0.5 weight %, based on the total cosmetic composition. In one embodiment, retinol is present in an amount of 0.05 to 0.5 weight %, based on the total cosmetic composition. In such a range, the effects derived from retinol can be achieved while inhibiting irritation.
(2) Oil gelling agent

The oil gelling agent is present in an amount of 0.5 to 20.0 weight %, based on the total cosmetic composition. When the oil gelling agent is present in an amount of 0.5 to 20.0 weight %, microneedles including a water soluble polymer can be uniformly and stably dispersed and combined in the oil gel including the cosmetic component, under the condition that the oil component includes silicone oil in an amount of 0 to 10.0 weight %, based on the total cosmetic composition.

The oil gelling agent is present in an amount of preferably 0.5 to 15.0 weight %, based on the total cosmetic composition. When the oil gelling agent is present in an amount of 0.5 to 15.0 weight %, a non-sticky and smooth cosmetic composition can be obtained. From the viewpoint of obtaining even more non-sticky and smooth cosmetic composition, the amount of oil gelling agent combined is preferably 1 to 15 weight %, and more preferably in the range of 5 to 15 weight %, based on the total cosmetic composition.

Examples of oil gelling agents included in the oil gel include 12-hydroxystearic acid; dextrin fatty acid esters such as dextrin palmitate and (palmitate/ethylhexanoate) dextrin; dibutyl lauroyl glutamide; dibutyl ethyl hexanoyl glutamide; sugar fatty acid esters; dibutyl ethyl hexanoyl glutamide; silica; and polymeric oil gelling agents such as hydrogenated (styrene/isoprene) copolymer and hydrogenated (styrene/butadiene) copolymer. Among the above, polymeric oil gelling agents, silica, and dextrin fatty acid esters are preferable from the viewpoint of stability; polymeric oil gelling agents and silica are more preferable; and polymeric oil gelling agents (e.g., hydrogenated (styrene/isoprene) copolymers) are particularly preferable.
(3) Oil component

Preferably, the oil component is present in an amount of 70 to 95 weight %, based on the total cosmetic composition. The oil gel can be obtained with the oil component in an amount of 70 weight % or more. With the amount of 95 weight % or less, a smooth gel that is not too viscous can be formed.

Examples of oil components included in the oil gel include hydrocarbon, ester oil, and silicone oil. It is preferable that the oil component includes at least one of hydrocarbon and ester oil.
(a) Hydrocarbon

Preferably, the hydrocarbon is present in an amount of 0 to 95 weight %, based on the total cosmetic composition. When the amount is out of this range, the oil gel may not be formed, or the gel may not be smooth.

In some embodiments, the hydrocarbon content is 40 to 95 weight % (e.g., 40 to 90 weight % or 45 to 85 weight %), based on the total cosmetic composition. In some embodiments, the oil component is free of hydrocarbon. In some embodiments, the hydrocarbon content is 0 to 45 weight % (e.g., 0 to 40 weight % or 0 to 30 weight %), based on the total cosmetic composition.

Examples of hydrocarbons constituting the oil component include liquid paraffin, squalane, squalene, paraffin, isohexadecane, mineral oil, polybutene, hydrogenated polybutene, and hydrogenated polydecene. Among the above, paraffin hydrocarbons such as isohexadecane and hydrogenated polydecene; and olefin hydrocarbons are preferable.
(b) Ester oil

Preferably, the ester oil is present in an amount of 0 to 95 weight %, based on the total cosmetic composition. When the amount is out of this range, the oil gel may not be formed, or the gel may not be smooth.

In some embodiments, the ester oil content is 0 to 45 weight % (e.g., 0 to 40 weight % or 0 to 35 weight %), based on the total cosmetic composition. In some embodiments, the oil component is free of ester oil. In some embodiments, the hydrocarbon content is 0 to 45 weight % (e.g., 0 to 40 weight % or 0 to 35 weight %), based on the total cosmetic composition.

Examples of ester oils constituting the oil component include dicaprylyl carbonate, tripropylene glycol dincopentanoate, isononyl isononanoate, isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, 12 cholesteryl hydroxystearate, cetyl ethylhexanoate, ethylene glycol di-2-ethylhexanoate, dipentaerythritol fatty acid esters, N-alkyl glycol monoisostearate, neopentyl dicaprate glycol, diisostearyl malate, glycerin di-2-heptylundecanoate, trimethylolpropane tri-2-ethylhexanoate, trimethylolpropane triisostearate, pentaerythrityl tetraethyl-hexanoate, triethyl hexanoin (tri-2-glyceryl ethylhexanoate), glycerin trioctanoate, glycerin triisopalmitate, trimethylolpropane triisostearate, cetyl-2-ethylhexanoate, 2-ethylhexyl palmitate, glyceryl trimyristate, tri-2-heptylundecanoic acid glyceride, castor oil fatty acid methyl ester, oleyl oleate, acetoglyceride, 2-heptyl undecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, and tri(caprylic/capric) glyceryl. Among the above, esters of fatty acids such as triester and tetraester such as pentaerythrityl tetraethylhexanoate and tri(caprylic/capric) glyceryl and polyhydric alcohols (preferably trivalent alcohol or tetravalent alcohol); and carbonic diesters such as dicaprylyl carbonate are preferable.
(c) Silicone oil

The oil component includes silicone oil in an amount of 0 to 10.0 weight %, based on the total cosmetic composition. Namely, the oil component is free of silicone oil or contains silicone oil in an amount of 10.0 weight % or less. When the oil component includes silicone oil in an amount of 0 to 10.0 weight %, microneedles including a water soluble polymer can be uniformly and stably dispersed and combined in the oil gel including the cosmetic component, under the condition that the oil gelling agent is present in an amount of 0.5 to 20.0 weight %, based on the total cosmetic composition.

Examples of silicone oils constituting the oil component include hydrogen dimethicone, caprylyl methicone, dimethicone, and cyclopentasiloxane.

### Typical Examples of Oil Component

In some embodiments, the oil component includes 0 to 95 weight % of hydrocarbon, 0 to 95 weight % of ester oil, and 0 to 10 weight % (e.g., 0 to 7 weight % or 0 to 5 weight %) of silicone oil, based on the total cosmetic composition.

In some embodiments, the oil component includes 40 to 95 weight % (e.g., 40 to 90 weight % or 45 to 85 weight %) of hydrocarbon, 0 to 45 weight % (e.g., 0 to 40 weight % or 0 to 35 weight %) of ester oil, and 0 to 10 weight % (e.g., 0 to 7 weight % or 0 to 5 weight %) of silicone oil, based on the total cosmetic composition.

In some embodiments, the oil component includes 0 to 45 weight % (e.g., 0 to 40 weight % or 0 to 30 weight %) of hydrocarbon, 50 to 95 weight % (e.g., 50 to 95 weight % or 60 to 95 weight %) of ester oil, and 0 to 10 weight % (e.g., 0 to 7 weight % or 0 to 5 weight %) of silicone oil, based on the total cosmetic composition.

In some embodiments, the oil component is free of silicone oil. In certain embodiments, the oil component includes 40 to 95 weight % (e.g., 40 to 90 weight % or 45 to 85 weight %) of hydrocarbon, and 0 to 45 weight % (e.g., 0 to 40 weight % or 0 to 35 weight %) of ester oil, based on the total cosmetic composition, and is free of silicone oil. In certain embodiments, the oil component includes 0 to 45 weight % (e.g., 0 to 40 weight % or 0 to 30 weight %) of hydrocarbon and 50 to 95 weight % (e.g., 50 to 95 weight % or 60 to 95 weight %) of ester oil, based on the total cosmetic composition, and is free of silicone oil. Such a formulation allows for a uniform and stable dispersion and combination of the microneedles.

In some embodiments, the oil component includes silicone oil. In certain embodiments, the oil component includes 40 to 95 weight % (e.g., 40 to 90 weight % or 45 to 85 weight %) of hydrocarbon, 0 to 45 weight % (e.g., 0 to 40 weight % or 0 to 35 weight %) of ester oil, and 1 to 10 weight % (e.g., 1 to 7 weight % or 1 to 5 weight % or 2 to 7 weight %) of silicone oil, based on the total cosmetic composition. In certain embodiments, the oil component includes 0 to 45 weight % (e.g., 0 to 40 weight % or 0 to 30 weight %) of hydrocarbon, 50 to 95 weight % (e.g., 50 to 95 weight % or 60 to 95 weight %) of ester oil, and 1 to 10 weight % (e.g., 1 to 7 weight % or 1 to 5 weight % or 2 to 7 weight %) of silicone oil, based on the total cosmetic composition. By combining silicone oil together with hydrocarbon and ester oil, a composition can be obtained in which microneedles are uniformly and stably dispersed and combined, while further reducing stickiness.
(4) Stabilizing agent

The stabilizing agent is present in an amount of preferably 0.01 to 5 weight %, based on the total cosmetic composition. When the amount is less than the above range, the effect cannot be obtained; and the amount exceeding the above range may cause irritation, etc. The stabilizing agent is present in an amount of, more preferably 0.01 to 1 weight %, and further preferably 0.05 to 1 weight %, based on the total cosmetic composition.

Examples of stabilizing agents included in the oil gel include preservatives, preservative auxiliaries, and antioxidants.

Examples of preservatives include phenoxyethanol.

Examples of preservative auxiliaries include bisabolol.

Examples of antioxidants include phenolic antioxidants; vitamin C antioxidants such as ascorbic acid, sodium ascorbate, and magnesium ascorbate; and tocopherol acetate. Examples of phenolic antioxidants include monophenolic antioxidants, bisphenolic antioxidants, and polymeric phenolic antioxidants. Specific examples include monophenolic antioxidants such as butylated hydroxyanisole (BHA) and dibutylhydroxytoluene (BHT); bisphenolic antioxidants such as 2,2′-methylenebis(4-methyl-6-tert-butylphenol), 2,2′-methylene bis(4-ethyl-6-tert-butylphenol), 4,4′-butylidene bis(3-methyl-6-tert butylphenol); and polymeric phenolic antioxidants such as tetrakis-[methylene-3-(3′,5′-di-tert-butyl 4′-hydroxyphenyl) propionate] methane, and α-tocopherol. Among the above, the phenolic antioxidant is preferably at least one of butylated hydroxyanisole (BHA) and dibutylhydroxytoluene (BHT).

In the case of using retinol as the cosmetic component, it is preferable that the oil gel includes a phenolic antioxidant (preferably, at least one of butylated hydroxyanisole (BHA) and dibutylhydroxytoluene (BHT)). Including phenolic antioxidants (preferably one or both of BHA and BHT) allows retinol in the cosmetic composition to be stable.

### 2. Microneedles

The microneedles include a water soluble polymer. The microneedles may have any shape with one or more sharp needle(s). The microneedles are manufactured by publicly known or conventional methods using a water soluble polymer. For example, the microneedles can be manufactured by methods such as pressing molten polymeric material in a mold, or by Droplet Extension (DEN) method in which the microneedles are shaped by dropping a small drop, abutting opposite patches together, and then stretching the patches. The microneedles are, for example, conical or pseudo-spherical in shape. Pseudo-spherical is a shape that tapers from a circular base to a tip, with the outer surface from the base to the tip resembling a trumpet. In some embodiments, the microneedles are conical or pseudo-spherical in shape, with a base diameter in the range of 200 μm to 3000 μm, the diameter of the tip in the range of 1 μm to 100 μm, and the height in the range of 100 μm to 1000 μm. In some embodiments, the microneedles are sphere microneedles with one or more sharp needles.

The microneedles preferably include 50 to 100 weight %, more preferably 75 to 100 weight %, even more preferably 90 to 100 weight %, and particularly preferably 100 weight % of the water soluble polymer, based on the total microneedles (weight).

The microneedles are preferably present in an amount of 0.01 to 10 weight %, based on the total cosmetic composition. When the amount is less than the above range, the microneedles cannot be combined uniformly, and when the amount exceeds the above range, smoothness of the formulation may decrease. From the viewpoint of both uniform combination and smoothness of the composition, the amount of the microneedles combined is more preferably 0.05 to 5 weight %, further preferably 0.1 to 3 weight %, and particularly preferably 0.1 to 1 weight %, based on the total cosmetic composition.

Examples of water soluble polymers for forming the microneedles include hyaluronic acid or a salt thereof (e.g., sodium hyaluronate), cross-linked hyaluronic acid or a salt thereof, collagen, sodium carboxymethyl cellulose, vinylpyrrolidone-vinylacetate copolymer, polyvinyl alcohol, polyvinylpyrrolidone, cross-linked polyethylene glycol, polyethylene glycol cross-linked polylactic acid or polyglycolic acid or polylactic-co-glycolic acid or polydioxanone, poly(styrene)-block poly(acrylic acid), polyethylene glycol-cross-linked PMVE/MA, cross-linked polyvinylpyrrolidone, sodium starch glycolate, cellulose, natural rubber, synthetic rubber, alginate, sodium polyacrylate, PEG cross-linked poly(methyl/vinyl ether/maleic acid) (PMVE/MA) and its esters, and PEG cross-linked poly(methyl/vinyl ether/maleic anhydride) (PMVE/MAH) and esters thereof.

Particularly, from the viewpoint of biocompatibility, it is preferable that the microneedles include hyaluronic acid or a salt thereof (e.g., sodium hyaluronate). The microneedles preferably contain 50 to 100 weight %, more preferably 75 to 100 weight %, even more preferably 90 to 100 weight %, and particularly preferably 100 weight % of hyaluronic acid or a salt thereof, based on the total microneedles (weight). In some embodiments, the microneedles are formed of hyaluronic acid or a salt thereof (i.e., including 100 weight % of hyaluronic acid or a salt thereof).

The microneedles may include a component other than the water soluble polymers. Examples of the component other than the water soluble polymers include, for example, the above cosmetic components and polymers other than the water-soluble polymer. In some embodiments, the microneedles include a water-soluble polymer (for example, hyaluronic acid or its salt) and a cosmetic ingredient (for example, retinol).

### B. Manufacturing Method

The cosmetic composition of this embodiment can be manufactured by publicly known or conventional methods. For example, the cosmetic composition of this embodiment can be manufactured by combining and agitating each component constituting the cosmetic composition.

### C. Effect

The cosmetic composition of this embodiment includes an oil gel including a cosmetic component, an oil gelling agent, and an oil component, and microneedles including a water soluble polymer, the microneedles being dispersed in the oil gel, the oil component including hydrocarbon, ester oil, and silicone oil in an amount of 0 to 10.0 weight %, based on the total cosmetic composition, and the oil gelling agent is present in an amount of 0.5 to 20.0 weight %, based on the total cosmetic composition. Therefore, a cosmetic composition can be obtained in which microneedles including a water soluble polymer are uniformly and stably dispersed and combined in an oil gel including a cosmetic component. Further, when such a cosmetic composition is applied to the skin, the microneedles open microscopic pores on the skin surface, which is expected to allow cosmetic components to penetrate into the stratum corneum and promote the effects of the cosmetic components. In addition, since the microneedles are formed using water soluble polymers, the effects of the water soluble polymers can be expected to be demonstrated.

Another cosmetic composition according to this embodiment includes, based on the total cosmetic composition: an oil gel including (a) 0.01 to 5.0 weight % of retinol, (b) 5 to 15.0 weight % of an oil gelling agent including at least one of a polymeric oil gelling agent and silica, and (c) an oil component including 0 to 10.0 weight % of silicone oil, 40 to 90 weight % of hydrocarbon, and 0 to 45 weight % of ester oil, and 0.1 to 10.0 weight % of microneedles formed of hyaluronic acid or a salt thereof, the microneedles being dispersed in the oil gel, where the cosmetic composition is anhydrous. Therefore, a cosmetic composition can be obtained in which microneedles formed of hyaluronic acid or a salt thereof are uniformly and stably dispersed and combined in an oil gel including retinol. Further, when such a cosmetic composition is applied to the skin, the microneedles open microscopic pores on the skin surface, which is expected to allow retinol to penetrate into the stratum corneum and promote the effects of retinol. In addition, since the microneedles are formed using hyaluronic acid and a salt thereof, moisturizing effects of hyaluronic acid and a salt thereof can be expected to be demonstrated.

### EXAMPLES

The present invention is further specifically described below based on the examples and comparative examples. The examples given below are some of the examples of the present invention and do not limit the present invention.

The materials used in the examples and comparative examples are as follows.

### (a) Microneedles

The microneedles used were manufactured using sodium hyaluronate (hereafter referred to as hyaluronic acid needles). The hyaluronic acid needles were manufactured by publicly known or conventional methods. The hyaluronic acid needles used in the examples and comparative examples contain 100 weight % of hyaluronic acid, based on the total hyaluronic acid needles.

FIG. 2 shows a schematic diagram of the hyaluronic acid needles used in the examples and comparative examples. The hyaluronic acid needles used in the examples and comparative examples are pseudo-spherical in shape, with a base diameter of 200 &micro;m to 3000 &micro;m, the tip diameter of 1 &micro;m to 100 &micro;m, and the height of 100 &micro;m to 1000 &micro;m.

### (b) Cosmetic Component

Retinol (product name Retinol50C manufactured by BASF) was used as the cosmetic component.

### (c) Oil Gelling Agent

Silica (product name AEROSIL 300 manufactured by Nippon Aerosil Co., Ltd.), dextrin (palmitate/ethylhexanoate) (product name Rheopearl TT2 manufactured by Chiba Flour Milling Co., Ltd.), and hydrogenated (styrene/isoprene) copolymer (product name BI-THIN 602 manufactured by Kraton Corporation) were used as the oil gelling agents.

### (d) Hydrocarbon

Hydrogenated polybutene (product name PARLEAM manufactured by NOF Corporation) and isohexadecane (product name ARLAMOL HD-LQ manufactured by CRODA) were used as the hydrocarbons forming the oil component.

### (e) Ester Oil

Pentaerythrityl tetraethylhexanoate (product name Cetiol PEEH4 SD manufactured by BASF), tri(caprylic/capric) glyceryl (product name Myritol 318 manufactured by BASF) and dicaprylyl carbonate (product name Cetiol CC manufactured by BASF) were used as the ester oils forming the oil component.

### (f) Silicone Oil

Dimethicone (manufactured by Shin-Etsu Silicone) was used as the silicone oil forming the oil component.

### (g) Stabilizing Agent

Phenoxyethanol (manufactured by Clariant), bisabolol (product name Dragosantol manufactured by Symrise), tocopherol acetate (manufactured by DSM), butylated hydroxyanisole (BHA) (manufactured by IMCD Benelux B.V.), and dibutylhydroxytoluene (BHT) (product name IONOL CP manufactured by Oxiris) were used as the stabilizing agents.

The content amount of each material included in the cosmetic compositions of the examples and comparative examples and the evaluation of the cosmetic compositions of the examples and comparative examples are shown in Table 1. The numbers in Table 1 indicate the weight percent of each material based on the total cosmetic composition.

TABLE 1

| | | | EXAMPLE | EXAMPLE | EXAMPLE | EXAMPLE | EXAMPLE | EXAMPLE | EXAMPLE |
|---|---|---|---|---|---|---|---|---|---|
| | | | 1 | 2 | 3 | 4 | 5 | 6 | 7 |
| Hyaluronic acid [symbol] | | | 0.5 | 0.5 | 0.5 | 0.5 | 0.5 | 0.5 | 0.5 |
| R [symbol] ol | | | 0.0 [symbol] | 0.0 [symbol] | 0.05 | 0.05 | 0.0 [symbol] | 0.0 [symbol] | 0.05 |
| O [symbol] | | [symbol] a | 5 | 10 | 5 | 20 | | 5 | 5 |
| | | [symbol] /ethyl [symbol] | | | | | | | |
| | | Hydrogenated [symbol] | | | | | 10 | | |
| | | copolymer | | | | | | | |
| [symbol] component | [symbol] | Hydrogenated [symbol] | 43.85 | 28.85 | 44.05 | 28.85 | 38.85 | 53.95 | |
| | | no [symbol] c [symbol] | 20 | 20 | 20 | 20 | 20 | 20 | |
| | [symbol] ol | Di [symbol] c [symbol] | 20 | 20 | 20 | 20 | 20 | 20 | |
| | | P [symbol] | 10 | 10 | 10 | 10 | 10 | | 10 |
| | | [symbol] caprylic/cap [symbol] glyceryl | 0.1 | 0. [symbol] | 0.1 | 0.1 | 0.1 | | [symbol] . [symbol] 5 |
| | Sil [symbol] | Dimo [symbol] | | [symbol] | | | | | |
| S [symbol] ap [symbol] | | [symbol] ethanol | 0.1 | 0. [symbol] | 0.1 | 0.1 | 0.1 | 0.1 | 0.1 |
| | | B [symbol] sa [symbol] | 0.1 | 0. [symbol] | 0.1 | 0.1 | 0.1 | 0.1 | 0.1 |
| | | [symbol] co [symbol] ol acetate | 0.1 | 0. [symbol] | 0.1 | 0.1 | 0.1 | 0.1 | 0.1 |
| | | BHA | 0.1 | 0. [symbol] | | 0.1 | 0.1 | 0.1 | 0.1 |
| | | BH [symbol] | 0.1 | 0. [symbol] | | 0.1 | 0.1 | 0.1 | 0.1 |
| Evaluation | | [symbol] eed stability | A | A | A | A | A | A | A |
| | | [symbol] ne stability | A | A | C | A | A | A | A |
| | | [symbol] ess | A | AA | A | C | A | A | A |
| | | Smoothness | A | A | A | C | A | A | A |

| | | | COMPARATIVE | COMPARATIVE | COMPARATIVE | COMPARATIVE |
|---|---|---|---|---|---|---|
| | | | EXAMPLE 1 | EXAMPLE 2 | EXAMPLE 3 | EXAMPLE 4 |
| Hyaluronic acid [symbol] | | | 0.5 | 0.5 | 0.5 | 0.5 |
| R [symbol] ol | | | 0.0 [symbol] | 0.0 [symbol] | 0.0 [symbol] | 0.05 |
| O [symbol] | | [symbol] a | | 1 [symbol] | | |
| | | [symbol] /ethyl [symbol] | | | [symbol] | |
| | | Hydrogenated [symbol] | | | | |
| | | copolymer | | | | |
| [symbol] component | [symbol] | Hydrogenated [symbol] | 28.85 | [symbol] 3.85 | 25.85 | 41.85 |
| | | no [symbol] c [symbol] | 20 | 20 | 20 | 20 |
| | [symbol] ol | Di [symbol] c [symbol] | 20 | 20 | 20 | 20 |
| | | P [symbol] | 10 | 10 | 10 | 10 |
| | | [symbol] caprylic/cap [symbol] glyceryl | 0.1 | 0.1 | 0.1 | 0.1 |
| | Sil [symbol] | Dimo [symbol] | 20 | 20 | 1 [symbol] | [symbol] |
| S [symbol] ap [symbol] | | [symbol] ethanol | 0.1 | 0.1 | 0.1 | 0.1 |
| | | B [symbol] sa [symbol] | 0.1 | 0.1 | 0.1 | 0.1 |
| | | [symbol] co [symbol] ol acetate | 0.1 | 0.1 | 0.1 | 0.1 |
| | | BHA | 0.1 | 0.1 | 0.1 | 0.1 |
| | | BH [symbol] | 0.1 | 0.1 | 0.1 | 0.1 |
| Evaluation | | [symbol] eed stability | C | C | C | C |
| | | [symbol] ne stability | A | A | A | A |
| | | [symbol] ess | AA | AA | AA | A |
| | | Smoothness | A | A | A | A |

[symbol]

indicates data missing or illegible when filed

The cosmetic compositions of the examples and comparative examples were manufactured as follows. First, hydrocarbon, ester oil, and silicone oil were combined to form an oil component. When the oil component is free of silicone oil, hydrocarbon and ester oil were combined. Retinol and stabilizing agent were added to the formed oil component, and thereafter an oil gelling agent was added and stirred until sufficiently thickened to obtain an oil gel containing retinol. After hyaluronic acid needles were added to the resulting oil gel, the oil gel was stirred for a specified time to obtain an anhydrous cosmetic composition.

The cosmetic compositions of the examples and comparative examples were evaluated as follows.

### Needle Stability

Stability of the hyaluronic acid needles in the cosmetic compositions was evaluated visually using the following criteria.
A: Hyaluronic acid needles are uniformly and stably dispersed throughout the oil gel.
B: Dispersion of hyaluronic acid needles in the oil gel is rather uneven.
C: Hyaluronic acid needles are precipitated in the oil gel.

### Retinol Stability

Stability of retinol in the cosmetic compositions was evaluated visually using the following criteria.
A: No change at all in color and smell of the oil gel.
B: Slight change in color and smell of the oil gel.
C: Significant change in color and smell of the oil gel.

### Non-Stickiness

Non-stickiness of the cosmetic compositions was evaluated by applying the cosmetic compositions to the skin of six subjects, and by bodily sensation, using the following criteria. A cosmetic composition is evaluated as sticky when the cosmetic composition applied to the skin remains thereon and feels sticky after a specified time has elapsed.
AA: 6 rated as non-sticky.
A: 4-5 rated as non-sticky.
B: 2-3 rated as non-sticky.
C: 0-1 rated as non-sticky.

### Smoothness

Smoothness of the cosmetic compositions was evaluated by applying the cosmetic compositions to the skin of six subjects, and by bodily sensation, using the following criteria. A cosmetic composition is evaluated as smooth when the cosmetic composition applied to the skin spreads well.
AA: 6 rated as smooth.
A: 4-5 rated as smooth.
B: 2-3 rated as smooth.
C: 0-1 rated as smooth.

The following points may be considered from the results of evaluation of the cosmetic compositions in the examples and comparative examples.

### Needle Stability

When the oil gel contains no oil gelling agent at all, or when the oil gel contains a large amount of silicone oil, stability of the hyaluronic acid needles is evaluated as low. In other words, when the oil gel includes an oil gelling agent and contains no silicone oil at all or contains only little silicone oil, stability of the hyaluronic acid needles is evaluated as high.

In Comparative Example 1, stability of the hyaluronic acid needles is rated C. This is because Comparative Example 1 contains no oil gelling agent at all, and contains more than 10.0 weight % (specifically, 20 weight %) of silicone oil. In Comparative Example 2, stability of the hyaluronic acid needles is rated C. This is because Comparative Example 2 contains 15 weight % of oil gelling agent, but contains more than 10.0 weight % (specifically, 20 weight %) of silicone oil. In Comparative Example 3, stability of the hyaluronic acid needles is rated C. This is because Comparative Example 3 contains 7 weight % of oil gelling agent, but contains more than 10.0 weight % (specifically, 12 weight %) of silicone oil. In Comparative Example 4, stability of the hyaluronic acid needles is rated C. This is because Comparative Example 4 only contains 10.0 weight % or less (specifically, 7 weight %) of silicone oil, but contains no oil gelling agent at all.

On the other hand, in Examples 1-7, the hyaluronic acid needles are uniformly and stably dispersed in the oil gel, and stability of the hyaluronic acid needle is rated A. This is because Examples 1-7 contain the oil gelling agent (specifically, 5.0 to 20 weight %), and only contain 10.0 weight % or less (specifically, 0 to 5.0 weight %) of silicone oil.

### Retinol Stability

Retinol stability is enhanced when the oil gel contains at least one of butylated hydroxyanisole (BHA) and dibutylhydroxytoluene (BHT), which prevent oxidization of retinol as the stabilizing agent.

In Example 3, stability of retinol is rated C. This is because Example 3 contains neither butylated hydroxyanisole (BHA) nor dibutylhydroxytoluene (BHT).

On the other hand, in Examples 1, 2, and 4-7, stability of retinol is rated A. This is because Examples 1, 2, and 4-7 contain butylated hydroxyanisole (BHA) and dibutylhydroxytoluene (BHT).

In Comparative Examples 1-4 as well, stability of retinol is rated A. This is because Examples 1-4 contain butylated hydroxyanisole (BHA) and dibutylhydroxytoluene (BHT).

While Table 1 only shows the case where stability of retinol is enhanced when both butylated hydroxyanisole (BHA) and dibutylhydroxytoluene (BHT) are contained, stability of retinol is enhanced when at least one of butylated hydroxyanisole (BHA) and dibutylhydroxytoluene (BHT) is contained.

### [Non-Stickiness> and <Smoothness>

When the oil gel contains no silicone oil at all or contains only little silicone oil and contains a large amount of oil gelling agent, non-stickiness and smoothness are evaluated as low.

In Example 4, non-stickiness and smoothness are both rated C. This is because Example 4 contains no silicone oil at all, and contains more than 15.0 weight % (specifically, 20 weight %) of oil gelling agent.

On the other hand, in Examples 1-3 and 5-7, non-stickiness and smoothness are both rated A or AA. This is because Examples 1-3 and 5-7 only contain 10.0 weight % or less (specifically, 0 to 5.0 weight %) of silicone oil but contain only 15.0 weight % or less (specifically, 5.0 to 15.0 weight %) of oil gelling agent.

In Comparative Examples 1-3 as well, non-stickiness and smoothness are both rated A or AA. This is because Comparative Examples 1-3 contain more than 10.0 weight % (specifically, 12.0 to 20 weight %) of silicone oil, and contain only 15.0 weight % or less (specifically, 0 to 15.0 weight %) of oil gelling agent.

In Comparative Example 4 as well, non-stickiness and smoothness are both rated A. This is because Comparative Example 4 only contains 10.0 weight % or less (specifically, 7 weight %) of silicone oil but contains only 15.0 weight % or less (specifically, 0 weight %) of oil gelling agent.

In the cosmetic compositions of Examples 1, 2, and 5-7, the hyaluronic acid needles are uniformly and stably dispersed in the oil gel. In addition, there is no change at all in the color and smell of the oil gel. When the cosmetic compositions of Examples 1, 2, and 5-7 are applied to the subject's skin, the subject feels smooth without stickiness. When the cosmetic compositions of Examples 1, 2, and 5-7 are applied to the subject's skin, the hyaluronic acid needles open microscopic pores on the skin surface, which is expected to allow retinol to penetrate into the stratum corneum and promote the effects of retinol. In addition, the moisturizing effect of hyaluronic acid is expected to be demonstrated.

While the present invention has been explained in details based on the embodiments and examples as above, the present invention is not limited thereto. It is obvious that modification and improvement within the scope of the technical idea of the present invention are possible for a person with ordinary skill in the art.

1. A cosmetic composition comprising:
an oil gel including a cosmetic component, an oil gelling agent, and an oil component, and
microneedles including a water soluble polymer, the microneedles being dispersed in the oil gel,
wherein the oil component includes hydrocarbon, ester oil, and silicone oil in an amount of 0 to 10.0 weight %, based on the total cosmetic composition, and
wherein the oil gelling agent is present in an amount of 0.5 to 20.0 weight %, based on the total cosmetic composition.

2. The cosmetic composition of claim 1, wherein the microneedles are present in an amount of 0.1 to 10.0 weight %, based on the total cosmetic composition.

3. The cosmetic composition of claim 1, wherein the cosmetic component is present in an amount of 0.01 to 5.0 weight %, based on the total cosmetic composition.

4. The cosmetic composition of claim 1, wherein the oil gelling agent is present in an amount of 5 to 15.0 weight %, based on the total cosmetic composition.

5. The cosmetic composition of claim 1, wherein the cosmetic component comprises at least one selected from a group consisting of an anti-wrinkle component, a whitening component, and a germicidal component.

6. The cosmetic composition of claim 5, wherein the anti-wrinkle component comprises retinol.

7. The cosmetic composition of claim 6, wherein the oil gel further comprises a stabilizing agent including a phenolic antioxidant.

8. The cosmetic composition of claim 1, wherein the oil gelling agent comprises at least one of a polymeric oil gelling agent and silica.

9. The cosmetic composition of claim 1, wherein the silicone oil comprises dimethicone.

10. The cosmetic composition of claim 1, wherein the hydrocarbon comprises at least one selected from a group consisting of olefin hydrocarbon and paraffin hydrocarbon.

11. The cosmetic composition of claim 1, wherein the ester oil comprises at least one selected from an ester of a fatty acid and a polyalcohol and carbonic diester.

12. The cosmetic composition of claim 1, wherein the hydrocarbon comprises at least one of hydrogenated polydecene and isohexadecane.

13. The cosmetic composition of claim 1, wherein the ester oil comprises at least one selected from dicaprylyl carbonate, pentaerythrityl tetraethyl-hexanoate, and tri(caprylic/capric) glyceryl.

14. The cosmetic composition of claim 1, wherein the oil gelling agent comprises at least one of hydrogenated (styrene/isoprene) copolymer and silica.

15. The cosmetic composition of claim 1, wherein the water soluble polymer comprises hyaluronic acid or a salt thereof.

16. A cosmetic composition comprising, based on the total cosmetic composition:
an oil gel comprising:
(a) 0.01 to 5.0 weight % of retinol,
(b) 5 to 15.0 weight % of an oil gelling agent including at least one of a polymeric oil gelling agent and silica, and
(c) an oil component including 0 to 10.0 weight % of silicone oil, 40 to 90 weight % of hydrocarbon, and 0 to 45 weight % of ester oil; and
0.1 to 10.0 weight % of microneedles formed of hyaluronic acid or a salt thereof, the microneedles being dispersed in the oil gel.
